(19) United States
(12) Patent Application Publication
(10) Pub. No.: US 2023/0154609 A1
(43) Pub. Date: May 18, 2023

(54) ELECTRONIC HEALTH RECORDS ANALYSIS USING ROBOTIC PROCESS AUTOMATION

(71) Applicant: International Business Machines Corporation, Armonk, NY (US)

(72) Inventors: Shikhar Kwatra, San Jose, CA (US); Sushain Pandit, Austin, TX (US); Iranna Dharmaraya Ankad, Bengaluru (IN); Vijay Ekambaram, Chennai (IN)

(21) Appl. No.: 17/455,450

(22) Filed: Nov. 18, 2021

(51) Int. Cl. G16H 50/20; G06N 3/00; G16H 10/60; G06V 30/19

(52) U.S. Cl. CPC G16H 50/20; G06N 3/008; G16H 10/60; G06V 30/19

(57) ABSTRACT

Provided is a method, system, and computer program product for analyzing an electronic health record (EHR) using robotic process automation (RPA). A processor may analyze an EHR associated with a user. The processor may identify, based on analyzing the EHR, one or more health parameters that are outside of a threshold range. The processor may determine a set of recommended actions that may be performed to cause the health parameter to fall within the threshold range. The processor may analyze activity data associated with the user. The processor may identify, based on the activity data, a set of known activities performed by the user. The processor may correlate the recommended actions with the known activities to identify a subset of personalized actions that are specific to the user. The processor may send the subset of personalized actions to the user.

### BACKGROUND

The present disclosure relates generally to the field of robotic process automation (RPA) and, more specifically, to analyzing electronic health records (EHRs) using RPA to generate personalized actionable alerts for a user.

Robotic process automation (RPA), also known as software robotics, uses automation technologies to mimic back-office tasks of human workers, such as extracting data, filling in forms, moving files, et cetera. It combines application programming interfaces (APIs) and user interface (UI) interactions to integrate and perform repetitive tasks between enterprise and productivity applications. By deploying scripts which emulate human processes, RPA tools complete autonomous execution of various activities and transactions across unrelated software systems.

### SUMMARY

Embodiments of the present disclosure include a method, system, and computer program product for analyzing electronic health records (EHRs) using robotic process automation (RPA) to generate personalized actionable alerts for a user. A processor may analyze an EHR associated with a user. The processor may identify, based on analyzing the EHR, one or more health parameters that are outside of a threshold range. The processor may determine a set of recommended actions that may be performed by the user to cause the health parameter to fall within the threshold range. The processor may analyze activity data associated with the user. The processor may identify, based on the activity, a set of known activities performed by the user. The processor may correlate the set of recommended actions with the set of known activities to identify a subset of personalized actions that are specific to the user. The processor may send the subset of personalized actions to the user.

The above summary is not intended to describe each illustrated embodiment or every implementation of the present disclosure.

### BRIEF DESCRIPTION OF THE DRAWINGS

The drawings included in the present disclosure are incorporated into, and form part of, the specification. They illustrate embodiments of the present disclosure and, along with the description, serve to explain the principles of the disclosure. The drawings are only illustrative of typical embodiments and do not limit the disclosure.

FIG. 1 illustrates a block diagram of an example robotic process automation system, in accordance with embodiments of the present disclosure.

FIG. 2 illustrates an example representation for extracting one or more health parameters from an electronic health record to identify a set of recommended actions, in accordance with embodiments of the present disclosure.

FIG. 3 illustrates an example representation for extracting activity data to generate a subset of personalized actions for a user, in accordance with embodiments of the present disclosure.

FIG. 4 illustrates a flow diagram of an example process for analyzing an electronic health record using robotic process automation to generate personalized actionable alerts for a user, in accordance with embodiments of the present disclosure.

FIG. 5 illustrates a high-level block diagram of an example computer system that may be used in implementing one or more of the methods, tools, and modules, and any related functions, described herein, in accordance with embodiments of the present disclosure.

FIG. 6 depicts a cloud computing environment in accordance with embodiments of the present disclosure.

FIG. 7 depicts abstraction model layers in accordance with embodiments of the present disclosure.

While the embodiments described herein are amenable to various modifications and alternative forms, specifics thereof have been shown by way of example in the drawings and will be described in detail. It should be understood, however, that the particular embodiments described are not to be taken in a limiting sense. On the contrary, the intention is to cover all modifications, equivalents, and alternatives falling within the spirit and scope of the disclosure.

### DETAILED DESCRIPTION

Aspects of the present disclosure relate to the field of robotic process automation (RPA) and, more particularly, to analyzing EHRs using RPA to generate personalized actionable alerts for a user. While the present disclosure is not necessarily limited to such applications, various aspects of the disclosure may be appreciated through a discussion of various examples using this context.

RPA is the use of software bots that can mimic the behaviour of human workers to automate repetitive, routine tasks. In many instances, it is highly challenging for hospitals, third party administrators, or even the individual users to maintain all of their online medical/health reports at one common location and derive actionable insights at periodic intervals from these reports. Often times, users undergo self-health check-ups instead of consulting a physician in person. In such scenarios, an individual user has to periodically review their medical reports manually and make sense of varying health parameters to decide whether to schedule an appointment with a doctor or not. In many instances, the user may forego or forget to follow-up with a health check-up, which could prove costly later if health conditions worsen.

Embodiments of the present disclosure include a system, computer-implemented method, and computer program product that are configured to utilize RPA software (e.g., RPA bot) to analyze a user's health records in order to generate a list of personalized actions for the user to perform to improve the user's health. The actions are specifically tailored for the user based on the user's own behavioral preferences to increase the likelihood that the user will complete the actions (i.e., adhere to the plan). In this way, the set of personalized actions attempt to maximize a correlation with the user's current behavioral patterns, while reducing the user's overall health risks.

In embodiments, the system may collect electronic health records (EHRs) on a periodic basis to determine the user's current health status. The EHRs may be collected from any type of EHR database or repository such as a cloud repository. For example, as the user undergoes any medical/health tests, subsequent laboratory reports are then uploaded to the cloud repository where they can be collected by the system.

In embodiments, the system utilizes an RPA bot to analyze the EHRs that are associated with the user. The RPA bot may utilize various computer vision techniques to extract contextual details related to the user's health from the EHRs. For example, the RPA bot may use optical character recognition (OCR) to analyze each EHR and identify one or more health parameters that are outside of a threshold range for the given parameter. This may be performed by identifying particular columns in a EHR report that contains both the actual result(s) and the normal range(s) for the given health parameter. For example, the RPA bot may analyze an EHR related to blood work of the user and identify that the user's sodium and glucose levels are both high and outside the normal range. In embodiments, the RPA bot may be trained to understand the logic related to the given types of reports in order to extract the health parameters.

In embodiments, the identified health parameters that are determined to be outside of the normal range may be analyzed using machine learning techniques to determine a set of recommended actions that may be performed by the user to cause the one or more health parameters to fall back within the threshold range. The machine learning techniques may be used to analyze the identified health parameter(s), various historical health parameters and ranges from past EHRs, and historic actions (e.g., such as exercise, dieting, health visits, etc.) for improving the health parameters to determine the set of recommended actions. In some embodiments, the machine learning techniques may be applied by a machine learning engine or system such as IBM Watson Health® (IBM Watson Health® based trademarks and logos are trademarks or registered trademarks of International Business Machines Corporation). For example, using IBM Watson Health®, the RPA bot may determine that the user should exercise more and eat less foods containing high amounts of salt and sugar in order for the given health parameters related to sodium and glucose to fall within the normal range. Further, based on the analysis of the EHR, the RPA bot may recommend the user schedule a follow-up doctor's visit if determined to be necessary based on the given results. The set of recommended actions will be sent to the RPA bot to correlate with the user's personal behaviors to determine the specific actions for the user based on their behavioral activity.

In embodiments, the RPA bot may collect/gather/receive various user activity data from multiple types of data sources. For example, the RPA bot may collect user activity data from one or more Internet of Things (IoT) devices associated with the user in order to determine the user's behavioral patterns (e.g., using a context vector). In another example, the RPA bot may collect user activity data from a website, such as a social media website or social calendar, to make determinations on what the user is doing on certain days (e.g., social media post indicating the user is going biking today). The RPA bot may analyze the activity and identify a set of known activities performed by the user. For example, the RPA bot may determine from the user's activity data that the user typically runs once a week (e.g., gathered from smart watch data/fitness application) and order's fast food 3 times/week (e.g., gathered from a food ordering software application or banking log obtained from the user's smart phone).

In embodiments, the RPA bot may correlate the set of recommended actions with the set of known activities to identify a subset of personalized actions that are specific to improving the user's health parameters. In embodiments, the RPA bot may include, exclude, and/or substitute various personalized actions based on the recommended actions and the user's known activities in order to maximize correlation to the user's current behavioral patterns while minimizing the user's overall health risk. For example, the RPA bot may augment the recommended actions (e.g., exercise more, eat less salty/sugary foods) with the known activities of the user (e.g., running once a week and ordering fast food 3 times/week) to identify that the user should run 3 times/week instead of once a week and order fast food only once a week to reduce both the salt/glucose levels. In another example, the RPA bot may suggest that the user increase the amount of time the user walks (e.g., going from 20 mins to 35 mins) and/or slightly alter the user's daily routine (e.g., suggest taking the stairs rather than the elevator) in order to improve the user's health. Further, the RPA bot may also suggest substituting health foods instead of fast food when ordering food delivery. In this way, the set of personalized actions attempt to maximize a correlation with the user's current behavioral patterns, while reducing the user's overall health risks. This can increase the likelihood that the user adheres to the health plan and makes the recommended changes to their lifestyle.

In embodiments, the RPA bot will send the subset of personalized actions to the user. The RPA bot may continuously monitor the user's activity data to determine if the user has performed the personalized set of actions. For example, the RPA bot can track user activity data to determine if the user has been running 3 times/week and/or if they have reduced ordering fast food. If the RPA bot identifies that the user has not performed the personalized actions, the RPA bot may send further alerts reminding the user to complete the actions. The RPA bot can mark that the personalized action has been completed if the user activity data indicates that it has been performed so the system will cease sending alerts until a subsequent analysis.

In embodiments, the RPA bot may continuously monitor the user's EHRs to determine if the user's health parameters have changed based on the implementation of the personalized actions. For example, the RPA bot may analyze new EHRs (that are uploaded to the cloud repository) to determine if a health parameter that was previously out of the threshold/normal range is now within the threshold range and notify the user of the change. In this way, the RPA bot may continuously monitor and assist the user in improving their health and/or health parameters.

In embodiments, the user(s) must opt into the system in order for the RPA bot to collect their information (e.g., EHRs, activity data, etc.), and the user may determine which other applications/users (e.g., RPA bot, IBM Watson Health, etc.) can utilize the collected data. For example, during an initialization process, the system may inform the user of the types of data that it will collect (e.g., user activity data, EHRs, past health parameters, etc.) and the reasons why the data is being collected. In these embodiments, the system will only start collecting the user information upon the user explicitly permitting the collection. Furthermore, the system may only collect the data that is necessary to provide the set of personalized action for improving the user's health. The collected data may be anonymized and/or encrypted while in use, and the data may only be maintained as needed for providing the recommendations. If the user chooses to opt out of the system, any user information previously collected may be permanently deleted.

The aforementioned advantages are example advantages, and not all advantages are discussed. Furthermore, embodiments of the present disclosure can exist that contain all, some, or none of the aforementioned advantages while remaining within the spirit and scope of the present disclosure.

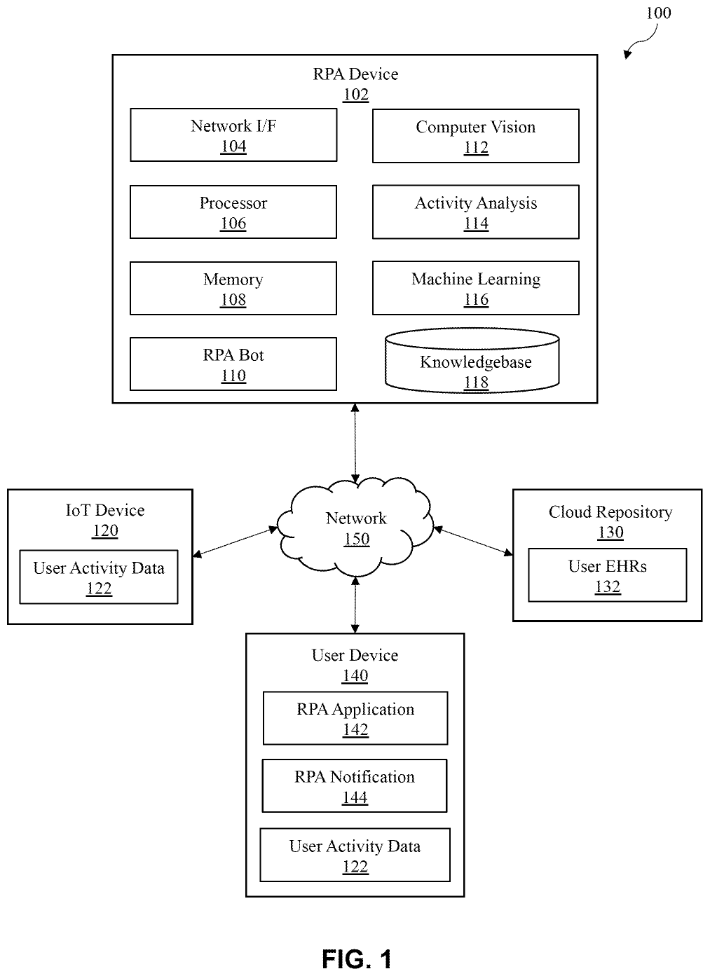

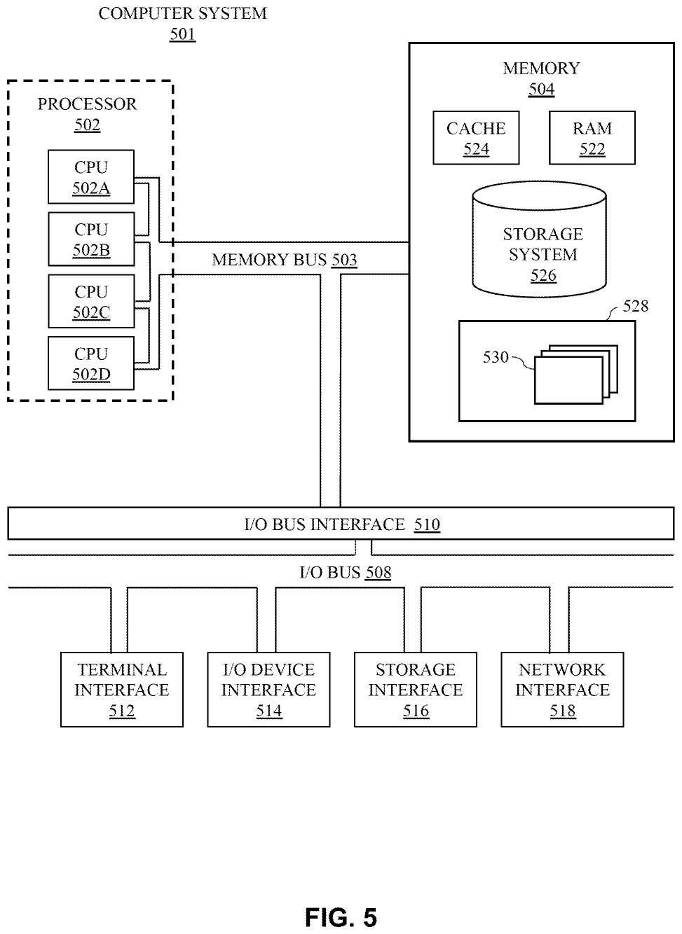

With reference now to FIG. 1, shown is a block diagram of robotic process automation (RPA) system 100, in accordance with embodiments of the present disclosure. In the illustrated embodiment, RPA system 100 includes RPA device 102 that is communicatively coupled to IoT device 120, cloud repository 130, and user device 140 via network 150. RPA device 102, IoT device 120, cloud repository 130, and user device 140 may be configured as any type of computer system and may be substantially similar to computer system 501 of FIG. 5.

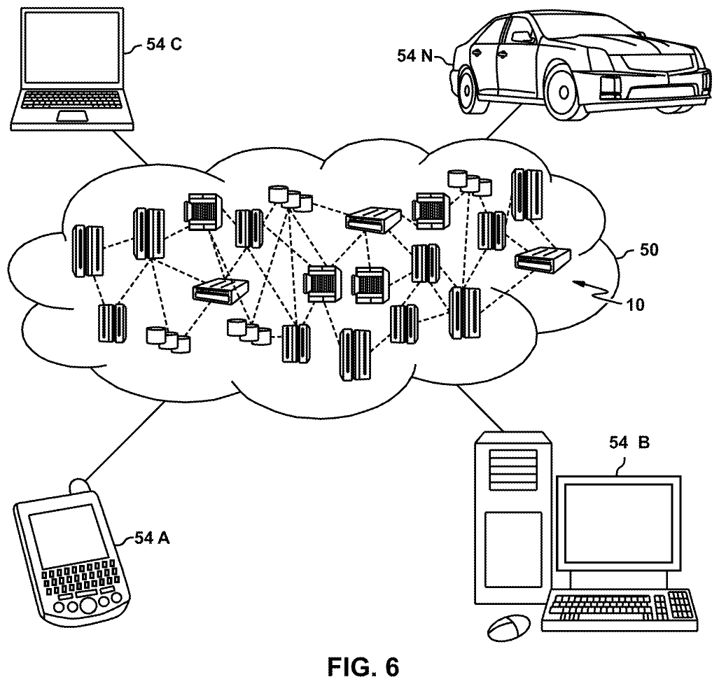

In embodiments, network 150 may be any type of communication network, such as a wireless network, edge computing network, a cloud computing network, or any combination thereof (e.g., hybrid cloud network/environment). Network 150 may be substantially similar to, or the same as, cloud computing environment 50 described in FIG. 6. Consistent with various embodiments, a cloud computing environment may include a network-based, distributed data processing system that provides one or more edge/network/cloud computing services. Further, cloud computing environment may include many computers (e.g., hundreds or thousands of computers or more) disposed within one or more data centers and configured to share resources over network 150.

In some embodiments, network 150 can be implemented using any number of any suitable communications media. For example, the network may be a wide area network (WAN), a local area network (LAN), a personal area network (PAN), an internet, or an intranet. In certain embodiments, the various systems may be local to each other, and communicate via any appropriate local communication medium. For example, RPA device 102 may communicate with IoT device 120, cloud repository 130, and user device 140 using a WAN, one or more hardwire connections (e.g., an Ethernet cable), and/or wireless communication networks. In some embodiments, the various systems may be communicatively coupled using a combination of one or more networks and/or one or more local connections. For example, in some embodiments RPA device 102 may communicate with cloud repository 130 using a hardwired connection, while communication between IoT device 120, user device 140, and RPA device 102 may be through a wireless communication network.

In embodiments, IoT device 120 and user device 140 may be any type of computing devices that generate user activity data 122. For example, user device 140 may be configured as a desktop, laptop, smartphone, etc., while IoT device 120 may be configured as a smart watch, smart speaker, smart camera, etc. It is noted these user device and IoT device examples are not meant to be limiting.

User activity data 122 may comprise any type of data or activity that can be used by RPA device 102 to determine known activities that are performed by the user. For example, user activity data 122 may include contextual data/metadata indicating that user performs various exercises (e.g., walking, running, biking, etc.) throughout a day week, month, year, etc. User activity data 122 may be collected from various software applications (e.g., fitness/health apps) located on or connected to IoT device 120. In another example, user activity data 122 may include textual data that indicates various payment history of the user. For example, user activity data 122 may include food purchasing history (e.g., online ordering/food ordering application) performed using user device 140. In some embodiments, the user activity data 122 may include data obtained from other data sources, such as websites (e.g., social media posts, social calendars, etc.) that can be used by the RPA bot to make determinations on known activities the user performs. For example, the user activity may include calendar information obtained from a website indicating the user is scheduled to attend swimming practice once a week.

In embodiments, user device 140 includes RPA application 142 which allows the RPA bot 110 to send alerts and/or personalized actions to the user. For example, the RPA application 142 may be a software applications that includes an interface that receives RPA notification 144.

Cloud repository 130 is configured to store/maintain existing user EHRs 132 associated the user. For example, EHRs 132 may include various laboratory records/tests, medical records, health reports, etc. that are associated with the user. EHRs 132 may be stored in a format that is readable by RPA bot 110. For example, EHRs 132 may be stored as a PDF file format where the RPA bot 110 can extract one or more health parameters and/or threshold ranges.

In embodiments, RPA device 102 includes network interface (I/F) 104, processor 106, memory 108, RPA bot 110, computer vision component 112, activity analysis component 114, machine learning component 116, and knowledgebase 118. In embodiments, RPA device 102 may be located on network 150 (e.g., located on a server or cloud network, etc.) or be configured as a standalone device that connects to the network 150. In embodiments, user device 140 and cloud repository 130 may also contain similar components (e.g., processors, memories, network UF, analysis components, etc.) as RPA device 102; however, for brevity purposes these components are not shown.

In embodiments, RPA device 102 utilizes RPA bot 110 to receive, collect, monitor, and/or analyze user EHRs 132 from cloud repository 130 and user activity data 122 from IoT device 120 and user device 140.

In embodiments, RPA bot 110 uses computer vision component 112 to analyze user EHRs 132 in order to extract and/or identify one or more health parameters that are not within a threshold/prescribed range. For example, RPA bot 110 may use optical character recognition (OCR) to analyze a given medical report or lab report to identify any parameters that are not in a prescribed range. This may be determined by analyzing various columns on the report that contain both the actual result related to the user and the prescribed range. As another example, charts or graphs may be analyzed using machine vision and/or modeled trained on different types of visual data representations (e.g., pie charts, line graphs, etc.) to extract the underlying data. If any parameter is found to be out of range, the RPA bot 110 will capture the given health parameter in a separate file for further analysis using machine learning component 116.

Machine learning component 116 will utilize the separate file containing the health parameters that are out of range to generate a set of recommended actions that may be performed by the user to cause the one or more health parameters to fall back within the prescribed range over time in response to implementing the actions. Machine learning component 116 may utilize various known relationships between the recommended actions and their effect on the given health parameter (e.g., the severity of the health parameter and the effectiveness that action has in changing/improving the health parameter) in order to identify the set of recommended actions. For example, the machine learning component 116 may identify that the user should perform more exercise to lower their cholesterol level to a normal range based on known relationships gathered from historic data that is stored/access from knowledgebase 118. The historic data may include previous user data and/or other users' data (e.g., crowdsourced data), medical data, and/or health data, medical papers, and/or other medical information that is used to make determinations for choosing appropriate recommended actions.

In some embodiments, machine learning component 116 may be configured as a machine learning engine, module, or system such as IBM Watson Health®. Machine learning component 116 can utilize machine learning and/or deep learning, where algorithms or models can be generated by performing supervised, unsupervised, or semi-supervised training on user activity data 122 and/or various data of knowledgebase 118 to improve the accuracy of determining the set of recommended actions. For example, the machine learning component 116 may determine over time that a first recommended action improves a first health parameter better than a second recommended action based on historical activity data. Accordingly, the machine learning component 116 may automatically recommended the first recommended action for improving the given health parameter. Machine learning algorithms can include, but are not limited to, decision tree learning, association rule learning, artificial neural networks, deep learning, inductive logic programming, support vector machines, clustering, Bayesian networks, reinforcement learning, representation learning, similarity/metric training, sparse dictionary learning, genetic algorithms, rule-based learning, and/or other machine learning techniques.

In embodiments, RPA bot 110 may collect user activity data 122 from both IoT device 120 and user device 140 to determine known activities/behaviors that the user performs. The RPA bot 110 may utilize activity analysis component 114 to perform analytics processing on the user activity data 122 to identify the known activities. For example, activity analysis component 114 may identify that the user typically bikes once a week based on user activity data gathered from a fitness application that is linked to the user's smart watch. In another example, the activity analysis component 114 may identify that the user eats breakfast at a diner every weekday based on an analysis of the user's food purchases from a financial application on their smart phone. Using this information, the RPA bot 110 may generate/identify a set of known activities that are performed by the user.

RPA bot 110 may correlate the set of recommended actions that were identified by the machine learning component 116 and the set of known activities performed by the user to identify a subset of personalized actions that are specific to the user. The subset of personalized actions may be performed by the user in order to improve the user's health and bring any health parameter that was initially out of range back within the prescribed range.

In embodiments, the RPA bot 110 may utilize a correlation score to determine which recommended action best matches the user's known activities/behaviors, and a health score to determine which activities maximize improvement in health and/or minimize health risks. For example, if the recommended action was to exercise more and the user performs known exercises such as running and cycling, but prefers running (e.g., user activity data showing the user runs often but only bikes once in a while), the RPA bot 110 will recommend that the user runs on a more consistent basis to improve their health parameter. In this way, the RPA bot 110 chooses personalized actions based on the user's behavior in order to increase the likelihood that the user will complete the actions.

The RPA bot 110 may send the set of personalized actions to the user device 140 via RPA application 142. In embodiments, the RPA bot 110 may continuously monitor the user activity data 122 to determine if the user has completed the personalized action(s). If the user has not completed the actions, the RPA bot 110 may send RPA notification 144 indicating the user should complete the action in order to improve their health and/or meet their health parameter ranges.

FIG. 1 is intended to depict the representative major components of RPA system 100. In some embodiments, however, individual components may have greater or lesser complexity than as represented in FIG. 1, components other than or in addition to those shown in FIG. 1 may be present, and the number, type, and configuration of such components may vary. Likewise, one or more components shown within RPA system 100 may not be present, and the arrangement of components may vary.

For example, while FIG. 1 illustrates an example RPA system 100 having a single RPA device 102, a single IoT device 120, a single user device 140, and a single cloud repository 130 that are communicatively coupled via a single network 150, suitable network architectures for implementing embodiments of this disclosure may include any number of RPA systems, RPA devices, IoT devices, user devices, cloud repositories, and networks. The various models, modules, systems, and components illustrated in FIG. 1 may exist, if at all, across a plurality of RPA systems, RPA devices, IoT devices, user devices, cloud repositories, and networks.

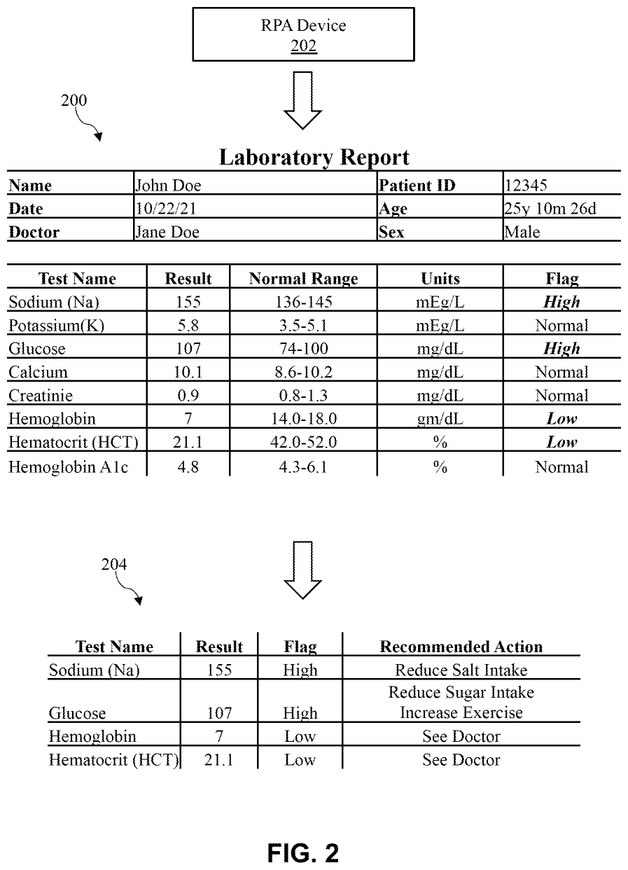

Referring now to FIG. 2, shown is an example representation of extracting one or more health parameters from an electronic health record (EHR) 200 to identify a set of recommended actions, in accordance with embodiments of the present disclosure.

In the illustrated embodiment, RPA device 202 is configured to extract and analyze contextual data from EHR 200. In embodiments, RPA device 202 may be substantially similar to RPA device 102 described in FIG. 1. EHR 200 is shown as an example laboratory report for a fictional user and is not meant to be limiting. It is contemplated that various forms of EHRs may be analyzed by the RPA device 202.

RPA device 202 may utilize one or more computer vision techniques to identify any health parameters that are not within a normal/threshold range. For example, RPA device 202 may utilize OCR to identify that the tests results for sodium, glucose, hemoglobin, and hematocrit (HCT) are all out of normal range for the user. These health parameters may be identified by comparing the test result to the given normal ranges, by the identification of the flag determination (e.g., high or low indication), or both. The identified health parameters that are determined to be out of range may be captured in a separate file 204 where they are further evaluated using a machine learning component (e.g., machine learning component 116 as detailed in FIG. 1).

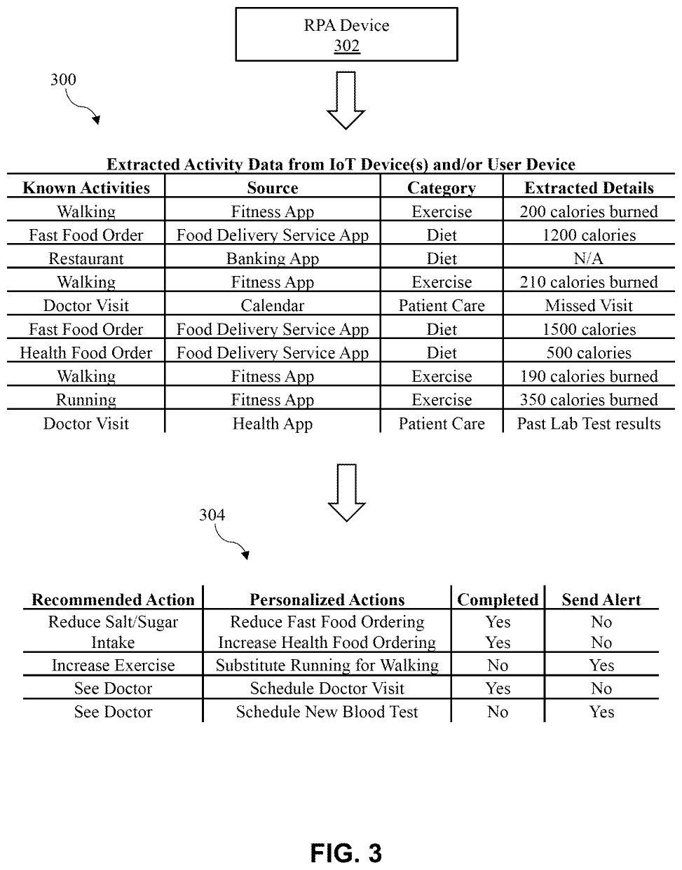

Using the machine learning component, RPA device 202 may identify a set of recommended actions that may be performed by the user to cause the one or more health parameters to fall within the normal range if implemented by over a time period. For example, based on the analyzed health parameters, the machine learning component may determine that the user should reduce sugar intake and increase their exercise regime to reduce their glucose test result such that it falls within the normal range. Further, the machine learning component may identify that the low hemoglobin count necessitates a doctor visit to be scheduled by the user. These recommended actions are then correlated with known activities that are performed by the user as described in FIG. 3 below.

Referring now to FIG. 3, shown is an example representation for extracting activity data 300 to generate a subset of personalized actions 304 for a user, in accordance with embodiments of the present disclosure.

In the illustrated embodiment, RPA device 302 is configured to extract and analyze activity data 300 that is gathered from one or more IoT device and/or user devices associated with the user. In embodiments, RPA device 302 may be substantially similar to RPA device 102 and/or RPA device 202 described in FIG. 1 and FIG. 2, respectively. Activity data 300 is shown as an example list of data for a fictional user and is not meant to be limiting. It is contemplated that additional types of activity data may be analyzed by the RPA device 302.

RPA device 302 identifies known activities that the user performs (e.g., walking, fast food ordering, running, doctor visit, etc.) from various contextual data obtained from the given data source (e.g., fitness app, food delivery app, calendar, banking app, etc.) on the IoT/user device(s). The known activities may be categorized based the type of activity. For example, walking and running activities may be categorized as exercise, while fast food order and health food order may be categorized by diet. Using the categories, the RPA device 302 may correlate the known activities with the recommended actions that were determined in FIG. 3. For example, the recommended action of “increase exercise” may be correlated with the known activities of walking and running. While the recommended action of reduce salt/sugar may be correlated with known activities that are categorized under diet. The RPA device 302 may use a correlation score to determine which known activities correlate best to the recommended actions.

Further, the RPA device 302 may utilize additional details from the extracted contextual data to generate a health risk score and/or a health improvement score to determine which actions may impact the user's health the most (e.g., maximize and/or minimize the user's health parameters) if implemented. For example, running may have a higher health improvement score than walking based on the amount of calories typically burned when each is performed by the user. In another example, fast food ordering may have a higher health risk score than health food ordering based upon the amount of calories consumed by the user for each given food type.

Using the correlation score and the health risk score/health improvement score, the RPA device 302 will generate a subset of personalized actions to be performed by the user in order to improve their health. For example, in order to reduce the user's salt/sugar intake, the RPA device 302 will identify that the user should reduce their fast food ordering and increase their health food ordering based on the given correlation/health risk scores. In another example, the RPA device 302 recommends that the user should substitute running for walking as the personalized action for increasing their exercise for reducing the blood sugar/glucose level. This is based on running having a higher health improvement score than walking based on calories burned. In this way, the RPA device 302 recommends personalized actions based on what types of activities the user has been doing by altering/modifying the frequency of each action in order to result in improvement of their health parameters. In this way, the RPA device allows the user to improve their health by recommending activities that they prefer.

Further, the RPA device 302 may continuously monitors and identify if the user has completed the given personalized activity. If the user has not completed the activity, the RPA device 302 may send a periodic alert notifying the user they have not completed the action.

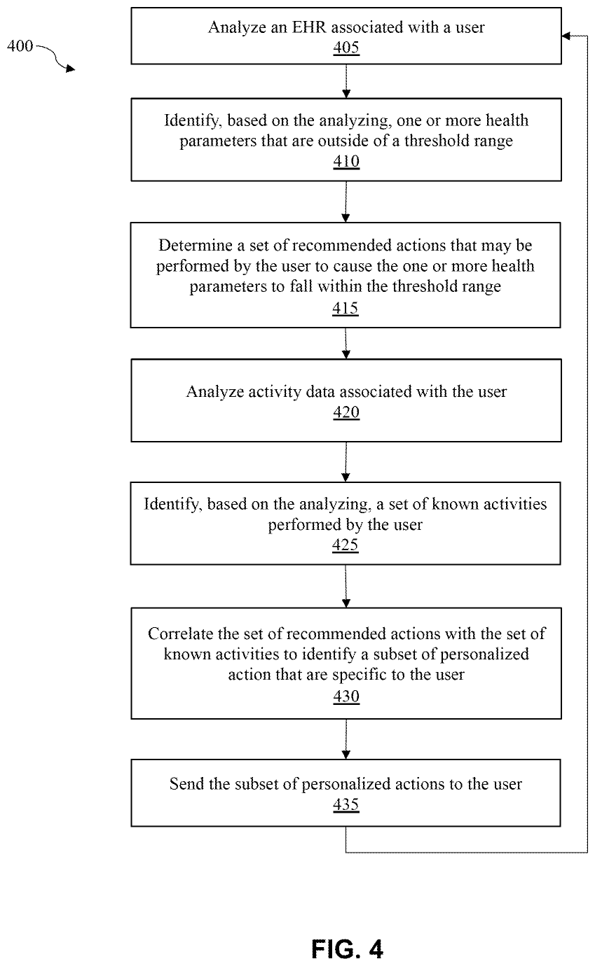

Referring now to FIG. 4, shown is a flow diagram of an example process 400 for analyzing an EHR using RPA to generate personalized actions for a user, in accordance with embodiments of the present disclosure. The process 400 may be performed by processing logic that comprises hardware (e.g., circuitry, dedicated logic, programmable logic, microcode, etc.), software (e.g., instructions run on a processor), firmware, or a combination thereof. In some embodiments, the process 400 is a computer-implemented process. In embodiments, the process 400 may be performed by processor 106 of RPA device 102 exemplified in FIG. 1.

The process 400 begins by analyzing an EHR associated with a user. This is illustrated at step 405. For example, RPA software/bot such as RPA bot 110 of FIG. 1 may be used to analyze the EHRs that are associated with the user. The RPA bot may utilize various computer vision techniques to extract contextual details related to the user's health from the EHR. For example, the RPA bot may use optical character recognition (OCR) to analyze the EHR and identify one or more health parameters that are outside of a threshold range for the given parameter.

The process 400 continues by identifying, based on analyzing the EHR, one or more health parameters that are outside of a threshold range. This is illustrated at step 410. For example, the RPA bot may analyze an EHR related to blood work (e.g., laboratory report 200 of FIG. 2) of the user and identify that the user's sodium and glucose levels are both high and outside the normal range. Further, the RPA bot may identify that the user's hemoglobin level is low and also out of the normal range.

The process 400 continues by determining a set of recommended actions that may be performed by the user to cause the one or more health parameters to fall within the threshold range. This is illustrated at step 415. In embodiments, the identified health parameters that are outside of the threshold range may be analyzed using machine learning techniques to determine the set of recommended actions that may be performed by the user to cause the one or more health parameters to fall within the threshold range. The machine learning techniques may be used to analyze the identified health parameter(s), various historical health parameters and ranges from past EHRs, and historic actions (e.g., such as exercise, dieting, health visits, etc.) for improving the health parameters to determine the set of recommended actions. In some embodiments, the machine learning techniques may be applied by IBM Watson Health®.

For example, using IBM Watson Health®, the RPA bot may determine that the user should exercise more and eat less foods with salt and sugar in order for the given health parameters to fall within the normal range. Further, based on the analysis of the EHR, the RPA bot may recommend the user schedule a follow-up doctor's visit to discuss possible remedies for the low hemoglobin level since this health parameter may require expert analysis.

The process 400 continues by analyzing activity data associated with the user. This is illustrated at step 420. The activity data associated with the user can be gathered/received from various types of data sources (e.g., one or more IoT devices, websites, social media blogs, calendars, etc.). For example, the RPA bot may collect user activity data from a smart watch and/or smart camera associated with the user to identify various types of activities the user performs.

The process 400 continues by identifying, based on analyzing the activity data, a set of known activities performed by the user. This is illustrated at step 425. For example, the RPA bot may determine from the user's activity data that the user typically walks 3 times/week, runs once a week (e.g., gathered from smart watch data/fitness application), orders fast food 3 times/week (e.g., gathered from a food ordering software application or banking log on the user's smart phone), and orders health food once a week.

The process 400 continues by correlating the set of recommended actions with the set of known activities to identify a subset of personalized actions that are specific to the user. This is illustrated at step 430. In embodiments, the subset of personalized actions may include personalized actions that exclude a previous known activity performed by the user, substitutes a first known activity performed by the user with a second known activity performed by the user, and/or substitutes a first known activity with a new activity.

For example, the recommended actions for lowering the user's salt and sugar levels were to exercise more and eat less salty/fatty foods. These recommended actions may be correlated to the user's known activities/behaviors (e.g., running, ordering fast food/health food) to identify the subset of personalized actions. For example, the subset of personalized actions may include an indication that the user should substitute walking with running at least 3 times/week instead of only running one in order to lower their glucose level and also to exclude ordering fast food, while increasing health food ordering. Further, the personalized action may include an indication that the user should schedule a follow up doctors visit with regard to the low hemoglobin level.

In embodiments, the subset of personalized action may be determined using a correlation score for each recommended action of the set of recommended actions with respect to each known activity in conjunction with a health risk score for each of the known activities. These scores may be used to determined which known activities meet both a minimum correlation score threshold and a minimum health risk threshold.

The process 400 continues by sending the subset of personalized actions to the user. This is illustrated at step 435. For example, the RPA bot may send the subset of personalized action to an application accessible on the user's smartphone to notify the user on what actions to perform to improve their health. In some embodiments, the RPA bot may continuously monitor the user activity data from the IoT devices to determine if the user has performed one or more of the subset of personalized actions.

For example, the RPA bot will track whether the user has been running the recommended amount of time in order to reduce the user's glucose levels. In another example, the RPA bot will monitor if the user has scheduled a follow-up doctor visit (e.g., analyzing calendar information or GPS coordinates of the user with respect to a doctor's office). In some embodiments, the RPA bot may automatically schedule the doctor's visit. If the RPA bot identifies the user has not completed the personalized activity, the RPA may send an alert to the user indicating the user has not performed the personalized action.

In embodiments, the RPA bot may receive an indication that the user has performed one or more personalized actions from the subset of personalized actions and mark the one or more personalized actions complete.

In some embodiments, the process 400 may return to step 405 to monitor/analyze any updated EHRs. For example, the RPA bot may analyze a second EHR associated with the user, where the second EHR is generated in response to the user completing at least one personalized action (e.g., follow-up doctor visit, additional bloodwork, etc.). The RPA bot may identify, based on analyzing the second EHR, that the one or more health parameters that were previously outside the threshold range are now within the given threshold range. In response, the RPA bot may notify the user that the one or more health parameters are within the threshold range. In this way, the RPA bot continuously monitors and updates the user regarding their health information.

Referring now to FIG. 5, shown is a high-level block diagram of an example computer system 501 that may be used in implementing one or more of the methods, tools, and modules, and any related functions, described herein (e.g., using one or more processor circuits or computer processors of the computer), in accordance with embodiments of the present disclosure. In some embodiments, the major components of the computer system 501 may comprise one or more CPUs 502, a memory subsystem 504, a terminal interface 512, a storage interface 516, an I/O (Input/Output) device interface 514, and a network interface 518, all of which may be communicatively coupled, directly or indirectly, for inter-component communication via a memory bus 503, an I/O bus 508, and an I/O bus interface 510.

The computer system 501 may contain one or more general-purpose programmable central processing units (CPUs) 502A, 502B, 502C, and 502D, herein generically referred to as the CPU 502. In some embodiments, the computer system 501 may contain multiple processors typical of a relatively large system; however, in other embodiments the computer system 501 may alternatively be a single CPU system. Each CPU 502 may execute instructions stored in the memory subsystem 504 and may include one or more levels of on-board cache. In some embodiments, a processor can include at least one or more of, a memory controller, and/or storage controller. In some embodiments, the CPU can execute the processes included herein (e.g., process 400 as described in FIG. 4). In some embodiments, the computer system 501 may be configured as RPA system 100 of FIG. 1.

System memory subsystem 504 may include computer system readable media in the form of volatile memory, such as random-access memory (RAM) 522 or cache memory 524. Computer system 501 may further include other removable/non-removable, volatile/non-volatile computer system data storage media. By way of example only, storage system 526 can be provided for reading from and writing to a non-removable, non-volatile magnetic media, such as a “hard drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory subsystem 504 can include flash memory, e.g., a flash memory stick drive or a flash drive. Memory devices can be connected to memory bus 503 by one or more data media interfaces. The memory subsystem 504 may include at least one program product having a set (e.g., at least one) of program modules that are configured to carry out the functions of various embodiments.

Although the memory bus 503 is shown in FIG. 5 as a single bus structure providing a direct communication path among the CPUs 502, the memory subsystem 504, and the I/O bus interface 510, the memory bus 503 may, in some embodiments, include multiple different buses or communication paths, which may be arranged in any of various forms, such as point-to-point links in hierarchical, star or web configurations, multiple hierarchical buses, parallel and redundant paths, or any other appropriate type of configuration. Furthermore, while the I/O bus interface 510 and the I/O bus 508 are shown as single units, the computer system 501 may, in some embodiments, contain multiple I/O bus interfaces 510, multiple I/O buses 508, or both. Further, while multiple I/O interface units are shown, which separate the I/O bus 508 from various communications paths running to the various I/O devices, in other embodiments some or all of the I/O devices may be connected directly to one or more system I/O buses.

In some embodiments, the computer system 501 may be a multi-user mainframe computer system, a single-user system, or a server computer or similar device that has little or no direct user interface, but receives requests from other computer systems (clients). Further, in some embodiments, the computer system 501 may be implemented as a desktop computer, portable computer, laptop or notebook computer, tablet computer, pocket computer, telephone, smart phone, network switches or routers, or any other appropriate type of electronic device.

It is noted that FIG. 5 is intended to depict the representative major components of an exemplary computer system 501. In some embodiments, however, individual components may have greater or lesser complexity than as represented in FIG. 5, components other than or in addition to those shown in FIG. 5 may be present, and the number, type, and configuration of such components may vary.

One or more programs/utilities 528, each having at least one set of program modules 530 may be stored in memory subsystem 504. The programs/utilities 528 may include a hypervisor (also referred to as a virtual machine monitor), one or more operating systems, one or more application programs, other program modules, and program data. Each of the operating systems, one or more application programs, other program modules, and program data or some combination thereof, may include an implementation of a networking environment. Programs/utilities 528 and/or program modules 530 generally perform the functions or methodologies of various embodiments.

It is understood in advance that although this disclosure includes a detailed description on cloud computing, implementation of the teachings recited herein are not limited to a cloud computing environment. Rather, embodiments of the present disclosure are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various search servers through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

Referring now to FIG. 6, illustrative cloud computing environment 50 is depicted. As shown, cloud computing environment 50 comprises one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

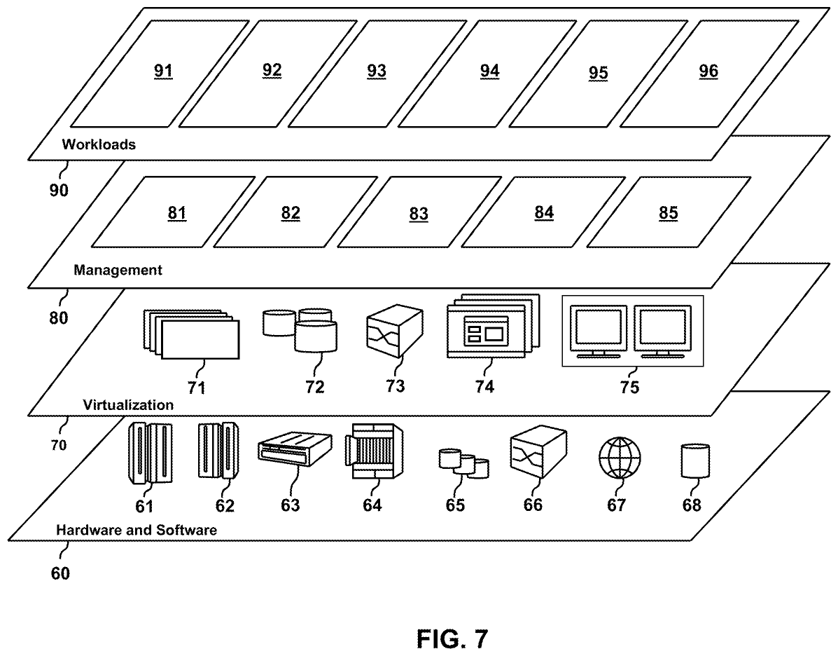

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and RPA bot software 68 in relation to the RPA system 100 of FIG. 1.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and RPA processing 96. For example, RPA system 100 of FIG. 1 may be configured to determine the set of personalized actions for the user using workloads layer 90.

As discussed in more detail herein, it is contemplated that some or all of the operations of some of the embodiments of methods described herein may be performed in alternative orders or may not be performed at all; furthermore, multiple operations may occur at the same time or as an internal part of a larger process.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the various embodiments. As used herein, the singular forms “a,” “an,” and “the” are intended to include the plural forms as well, unless the context clearly indicates otherwise. It will be further understood that the terms “includes” and/or “including,” when used in this specification, specify the presence of the stated features, integers, steps, operations, elements, and/or components, but do not preclude the presence or addition of one or more other features, integers, steps, operations, elements, components, and/or groups thereof. In the previous detailed description of example embodiments of the various embodiments, reference was made to the accompanying drawings (where like numbers represent like elements), which form a part hereof, and in which is shown by way of illustration specific example embodiments in which the various embodiments may be practiced. These embodiments were described in sufficient detail to enable those skilled in the art to practice the embodiments, but other embodiments may be used and logical, mechanical, electrical, and other changes may be made without departing from the scope of the various embodiments. In the previous description, numerous specific details were set forth to provide a thorough understanding the various embodiments. But, the various embodiments may be practiced without these specific details. In other instances, well-known circuits, structures, and techniques have not been shown in detail in order not to obscure embodiments.

As used herein, “a number of” when used with reference to items, means one or more items. For example, “a number of different types of networks” is one or more different types of networks.

When different reference numbers comprise a common number followed by differing letters (e.g., 100a, 100b, 100c) or punctuation followed by differing numbers (e.g., 100-1, 100-2, or 100.1, 100.2), use of the reference character only without the letter or following numbers (e.g., 100) may refer to the group of elements as a whole, any subset of the group, or an example specimen of the group.

Further, the phrase “at least one of,” when used with a list of items, means different combinations of one or more of the listed items can be used, and only one of each item in the list may be needed. In other words, “at least one of” means any combination of items and number of items may be used from the list, but not all of the items in the list are required. The item can be a particular object, a thing, or a category.

For example, without limitation, “at least one of item A, item B, or item C” may include item A, item A and item B, or item B. This example also may include item A, item B, and item C or item B and item C. Of course, any combinations of these items can be present. In some illustrative examples, “at least one of” can be, for example, without limitation, two of item A; one of item B; and ten of item C; four of item B and seven of item C; or other suitable combinations.

Different instances of the word “embodiment” as used within this specification do not necessarily refer to the same embodiment, but they may. Any data and data structures illustrated or described herein are examples only, and in other embodiments, different amounts of data, types of data, fields, numbers and types of fields, field names, numbers and types of rows, records, entries, or organizations of data may be used. In addition, any data may be combined with logic, so that a separate data structure may not be necessary. The previous detailed description is, therefore, not to be taken in a limiting sense.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Although the present invention has been described in terms of specific embodiments, it is anticipated that alterations and modification thereof will become apparent to the skilled in the art. Therefore, it is intended that the following claims be interpreted as covering all such alterations and modifications as fall within the true spirit and scope of the invention.

What is claimed is:

1. A method comprising:
analyzing an electronic health record (EHR) associated with a user;
identifying, based on analyzing the EHR, one or more health parameters that are outside of a threshold range;
determining a set of recommended actions that may be performed by the user to cause the one or more health parameters to fall within the threshold range;
analyzing activity data associated with the user;
identifying, based on analyzing the activity data, a set of known activities performed by the user;
correlating the set of recommended actions with the set of known activities to identify a subset of personalized actions that are specific to the user; and
sending the subset of personalized actions to the user.

2. The method of claim 1, wherein analyzing the EHR associated with the user is performed using one or more computer vision techniques.

3. The method of claim 2, wherein the one or more computer vision techniques include optical character recognition.

4. The method of claim 1, wherein the method is performed using robotic process automation (RPA).

5. The method of claim 1, wherein the activity data associated with the user is received from one or more Internet of Things (IoT) devices.

6. The method of claim 1, wherein the subset of personalized actions sent to the user comprises at least one personalized action selected from the group of actions consisting of:
recommending a previous activity performed by the user;
excluding a previous activity performed by the user;
substituting a first activity previously performed by the user with a second activity previously performed by the user; and
substituting the first activity previously performed by the user with a new activity.

7. The method of claim 1, wherein correlating the set of recommended actions with the set of known activities to identify the subset of personalized actions that are specific to the user comprises:
determining a correlation score for each recommended action of the set of recommended actions with respect to each known activity of the set of known activities;
determining a health risk score for each of the known activities of the set of known activities; and
identifying the subset of personalized actions by determining which known activities meet a minimum correlation score threshold and a minimum health risk threshold.

8. The method of claim 1, further comprising:
monitoring the activity data to determine if the user has performed one or more of the subset of personalized actions; and
sending, in response to determining that at least one personalized actions from the subset of personalized actions has not been performed, an actionable alert to the user indicating the user has not performed the personalized action.

9. The method of claim 1, further comprising:
receiving an indication that the user has performed one or more personalized actions from the subset of personalized actions; and
marking the one or more personalized actions complete.

10. The method of claim 1, further comprising:
analyzing a second EHR associated with the user, wherein the second EHR is generated in response to the user completing at least one personalized action;
identifying, based on analyzing the second EHR, that the one or more health parameters that were previously outside the threshold range are now within the given threshold range; and
notifying the user that the one or more health parameters are within the threshold range.

11. A system comprising:
a processor; and
a computer-readable storage medium communicatively coupled to the processor and storing program instructions which, when executed by the processor, cause the processor to perform a method comprising:
analyzing an electronic health record (EHR) associated with a user;
identifying, based on analyzing the EHR, one or more health parameters that are outside of a threshold range;
determining a set of recommended actions that may be performed by the user to cause the one or more health parameters to fall within the threshold range;
analyzing activity data associated with the user;
identifying, based on analyzing the activity data, a set of known activities performed by the user;
correlating the set of recommended actions with the set of known activities to identify a subset of personalized actions that are specific to the user; and
sending the subset of personalized actions to the user.

12. The system of claim 11, wherein the method is performed using robotic process automation (RPA).

13. The system of claim 11, wherein the subset of personalized actions sent to the user comprises at least one personalized action selected from the group of actions consisting of:
recommending a previous activity performed by the user;
excluding a previous activity performed by the user;
substituting a first activity previously performed by the user with a second activity previously performed by the user; and
substituting the first activity previously performed by the user with a new activity.

14. The system of claim 11, wherein correlating the set of recommended actions with the set of known activities to identify the subset of personalized actions that are specific to the user comprises:
determining a correlation score for each recommended action of the set of recommended actions with respect to each known activity of the set of known activities;
determining a health risk score for each of the known activities of the set of known activities; and
identifying the subset of personalized actions by determining which known activities meet a minimum correlation score threshold and a minimum health risk threshold.

15. The system of claim 11, wherein the method performed by the processor further comprises:
monitoring the activity data to determine if the user has performed one or more of the subset of personalized actions; and
sending, in response to determining that at least one personalized actions from the subset of personalized actions has not been performed, an actionable alert to the user indicating the user has not performed the personalized action.

16. The system of claim 11, wherein the method performed by the processor further comprises:
analyzing a second EHR associated with the user, wherein the second EHR is generated in response to the user completing at least one personalized action;
identifying, based on analyzing the second EHR, that the one or more health parameters that were previously outside the threshold range are now within the given threshold range; and
notifying the user that the one or more health parameters are within the threshold range.

17. A computer program product comprising a computer-readable storage medium having program instructions embodied therewith, the program instructions executable by a processor to cause the processor to perform a method comprising:
analyzing an electronic health record (EHR) associated with a user;
identifying, based on analyzing the EHR, one or more health parameters that are outside of a threshold range;
determining a set of recommended actions that may be performed by the user to cause the one or more health parameters to fall within the threshold range;
analyzing activity data associated with the user;
identifying, based on analyzing the activity data, a set of known activities performed by the user;
correlating the set of recommended actions with the set of known activities to identify a subset of personalized actions that are specific to the user; and
sending the subset of personalized actions to the user.

18. The computer program product of claim 17, wherein the method is performed using robotic process automation (RPA).

19. The computer program product of claim 17, wherein the subset of personalized actions sent to the user comprises at least one personalized action selected from the group of actions consisting of:
recommending a previous activity performed by the user;
excluding a previous activity performed by the user;
substituting a first activity previously performed by the user with a second activity previously performed by the user; and
substituting the first activity previously performed by the user with a new activity.

20. The computer program product of claim 17, wherein correlating the set of recommended actions with the set of known activities to identify the subset of personalized actions that are specific to the user comprises:
determining a correlation score for each recommended action of the set of recommended actions with respect to each known activity of the set of known activities;
determining a health risk score for each of the known activities of the set of known activities; and
identifying the subset of personalized actions by determining which known activities meet a minimum correlation score threshold and a minimum health risk threshold.
